(19) United States
(12) Patent Application Publication
(10) Pub. No.: US 2017/0312555 A1
(43) Pub. Date: Nov. 2, 2017

(54) RADIO FREQUENCEY POWERED RESISTIVE CHEMICAL SENSOR

(71) Applicant: Honeywell International Inc., Morris Plains, NJ (US)

(72) Inventors: Brian Keith Olmsted, Richfield, MN (US); Christopher Scott Larsen, Plymouth, MN (US)

(21) Appl. No.: 15/336,601

(22) Filed: Oct. 27, 2016

Related U.S. Application Data
(60) Provisional application No. 62/328,292, filed on Apr. 27, 2016.

(51) Int. Cl. A62B 9/00; G01N 27/12; A62B 18/02; A62B 23/02; G01N 33/00

(52) U.S. Cl. CPC A62B 9/006; G01N 33/0062; G01N 33/0031; A62B 18/02; A62B 23/02; G01N 27/122

(57) ABSTRACT

A gas sensor includes a chemiresistor having responsive to a specific gas vapor to be sensed such that a resistance of the chemiresistor changes responsive to exposure to the specific gas vapor. A data collection circuit is coupled to the chemiresistor to sense the change in resistance responsive to the specific gas vapor. An antenna is coupled to the data collection circuit to receive power from an RF interrogation signal, power the data collection circuit with the received power, and transmit a signal from the data collection circuit representative of the resistance of the chemiresistor.

### RELATED APPLICATION

This application claims priority to U.S. Provisional Application Ser. No. 62/328,292 (entitled Radio Frequency Powered Resistive Chemical Sensor, filed Apr. 27, 2016) which is incorporated herein by reference.

### BACKGROUND

There are significant technical problems in sensing gases or the noxious vapors of solvent chemicals in the workplace and in consumer applications. These challenges include:

(1) An inability to differentiate between different kinds of vapors or gases

(2) Sensors available on the market today have power requirements that are prohibitive for many gas sensing applications

(3) The cost of many gas sensing systems are prohibitive for incorporation into hardware that may or may not be designed to be disposable, thus limiting use case scenarios.

Additionally, as a specific application, end of service life products presently available for Air Purifying Respirators (APR's) that are designed to filter solvent vapors are not generally reliable enough to replace calculation tables. Consequently, users do not know when to replace the filtration cartridges installed in the APR, and must rely on calculation tables and poor estimates of gas concentration for a given unknown environment.

### SUMMARY

A gas sensor includes a chemiresistor having responsive to a specific gas vapor to be sensed such that a resistance of the chemiresistor changes responsive to exposure to the specific gas vapor. A data collection circuit is coupled to the chemiresistor to sense the change in resistance responsive to the specific gas vapor. An antenna is coupled to the data collection circuit to receive power from an RF interrogation signal, power the data collection circuit with the received power, and transmit a signal from the data collection circuit representative of the resistance of the chemiresistor.

A method of sensing gas includes exposing to air, a chemiresistor having a signaling chemical responsive to a specific gas vapor to be sensed such that a resistance of the chemiresistor changes responsive to exposure to the specific gas vapor, sensing a specific gas vapor in the air via a data collection circuit coupled to the chemiresistor to sense the resistance that changes responsive to exposure to the specific gas vapor, and receiving power via an RF interrogation signal at an antenna coupled to the data collection circuit and using the received power to transmit a signal from the data collection circuit representative of the resistance of the chemiresistor.

An air purifying respirator includes a filtration cartridge, a mask coupled to the filtration cartridge, the mask configured to provide an air path from ambient to a wearer of the mask through the filtration cartridge, and an end of service life gas sensor. The end of service life gas sensor may include a chemiresistor having a signaling chemical responsive to a specific gas vapor to be sensed such that a resistance of the chemiresistor changes responsive to exposure to the specific gas vapor, a data collection circuit coupled to the chemiresistor to sense the change in resistance responsive to exposure to the specific gas vapor, and an antenna coupled to the data collection circuit to receive power from an RF interrogation signal, power the data collection chipset with the received power, and transmit a signal from the data collection chipset representative of the resistance of the chemiresistor.

### BRIEF DESCRIPTION OF THE DRAWINGS

FIG. 1 is a block diagram of a radio frequency powered resistive chemical sensor system according to an example embodiment.

FIG. 2 is a block schematic diagram of a CCS-S sensing array.

FIG. 3 is a flowchart illustrating a chemiresistor sensor fabricating process according to an example embodiment.

FIGS. 4 and 5 are diagrams illustrating specific electrostatic potential modulations via adsorptive interactions for discerning different gas vapors according to an example embodiment.

FIG. 6 is a graph illustrating resistance ratio responses for two different chemiresistors wherein one is responsive to water vapor and the other is not responsive to water and is responsive to gas vapor according to an example embodiment.

FIG. 7 is a graph illustrating resistance ratio responses for two different chemiresistors when exposed concurrently to water vapor and THF according to an example embodiment.

FIG. 8 is a partially exploded view of a respirator with cartridges having end of service life indicators according to an example embodiment.

FIG. 9 is a diagram illustrating various selector chemicals having different gas sensitivity according to an example embodiment.

FIG. 10 is a block schematic diagram of circuitry for implementing embodiments and methods according to example embodiments.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

Demand for low power sensors is increasing at a tremendous rate. From structural wireless networks to body-worn systems, there are consistent needs for low cost, low power sensors. Most wireless sensors available today are powered sensors that will have a finite life and will need periodic replacement. Servicing sensor nodes can be challenging and expensive. Additionally, even the modest cost of an inexpensive battery may negate the economic justification for a product. Wireless sensors capable of being powered and queried from a distance would enable significant reduction of installation and maintenance costs and a gamut of sensing applications can be addressed by implementing minor modifications to the sensing element. Such a sensor could rapidly find use as an End of Service Life Indicator (ESLI) for Respirators.

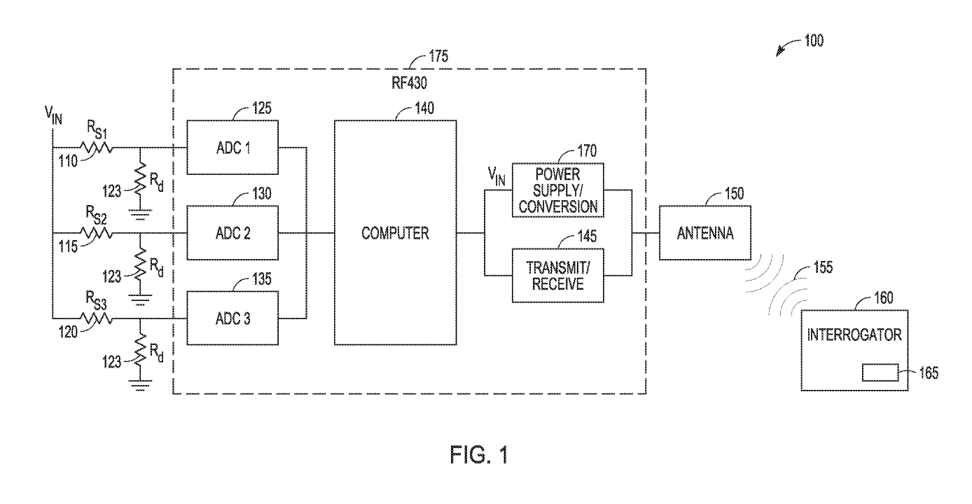

FIG. 1 is a block diagram of a radio frequency powered resistive chemical sensor system 100 according to an example embodiment. In one embodiment an array of chemiresistors R_{S1}-R_{S3} are chemiresistors 110, 115, and 120 acting as sensing elements. Other sensing elements that operate at low power may be used in further embodiments. Analog to digital convers (ADCs) 125, 130, 135 are coupled to respective chemiresistors 110, 115, and 120. The ADCs provide digital signals that change based on the change in resistance of the chemiresistors responsive to exposure to various chemicals.

Resistors 123 are coupled between the chemiresistors/ADC connection and ground, forming a voltage divider such that the voltage across resistors 123 is received by the ADCs, and varies responsive to changes in resistance of the chemiresistors. The voltage is a function of the resistance ratios between the chemical responsive chemiresistors and the reference resistors 123, which may nominally be substantially equal, or otherwise selected to provide a voltage within an input range of the ADCs. In one embodiment, the resistance is proportional to electrostatic interaction of the chemiresistors with the gas and hence the resistance is also proportional to the gas concentration.

A computer 140 is coupled to the ADCs and receives the digital signals. The computer 140 may be a low power programmed circuit that provides data representative of sensed chemicals for transmission via a transceiver 145, which is coupled to an antenna 150. The antenna 150 may receive interrogation signals 155 from a remote interrogation device 160, which may be powered by a button type battery 165 or other battery or power source. The interrogation signals 155 provide power to a power supply/conversion circuit 170. Power supply circuit 170 may convert the received power to a voltage, Vᵢₙ, which is provided to the computer 140 and chemiresistors 110, 115, 120. In one embodiment, an RF chip, such as an RF430 from Texas Instruments, may be used to provide the elements within a broken line box indicated at 175. In various embodiments, the sensor system 100 may be implemented as a data collection circuit in the form of a chipset.

In one embodiment, the chemiresistors may be formed in the following manner. Carbon-based conductive substrates (CCS) are paired with gaseous vapor selector chemicals (S) and are solventlessly blended such that electrostatic coupling is achieved between the CCS and the S chemicals. Polyaryl compounds strongly adsorb to CCS's through dispersive forces of conjugated π bonding systems and by choosing molecules with polyaryl structures and various pendant functionalities, selective gas sensing can be achieved. To accomplish the coupling, CCS's may be mechanically milled with a battery of various S candidates to achieve adsorptively coupled composites as sensing materials, also referred to as bonded or otherwise associated signaling chemicals. The adsorptively coupled composites synergistically enable chemiresistive sensing when the S compound selectively adsorbs a targeted gaseous vapor species.

Deposition of the composite sensing media may be done by a dry mechanical transfer process, a method that is effective, facile, and inexpensive. The transfer process utilizes mechanical compaction of the composite sensing material into a pelletized form that is dry transferred to a substrate bridging two deposited electrodes.

Final gas vapor discrimination may be performed via an algorithm based on Principle Component Analysis (PCA) using relative CCS:S combinations and relative sensor responsivities in one embodiment.

The radio frequency powered chemical sensor system includes this new type of chemiresistor. The operating principle of the chemiresistor relies on the strong intermolecular forces of physisorbed and chemisorbed “signaling chemicals” to a surface of a carbon-based substrate and a conjugated pi-bonding network of the sp2 carbon-carbon bond network characterizing these substrates.

Additionally, high-aspect ratio forms of carbon, such as single and multi-walled carbon nanotubes, enhance the sensitivity and conduction of the substrate through percolation theory principles. The aspect ratio of the nanotubes (the length) impacts the range of operating resistances that may be selectively engineered. Graphene film based substrates may be used in further embodiments.

The “signaling chemical” is chosen so that a part of the molecule has a high propensity for adsorption and van der Waals bonding to the carbon substrate, and possesses a functional group capable of strong electrostatic interaction with specific gas vapors. Thus, gas vapors adsorb selectively to the pendant functional group and modulate the electrostatic potential of the complexed chemiresistor, resulting in the modulation of the substrate's resistance.

The chemiresistor serves as an unpowered, resistive gas sensor integratable with an ADC (Analog to Digital Converter) circuit. Such a chipset may be strictly powered by harnessing, for example, the field from an RF reader, such as an RFID reader, as it interrogates the sensor. As described above with respect to FIG. 1, the resistive gas sensor is coupled to an analog to digital converter on a new class of microcontroller available that is powered solely through the RFID reader's field. The microcontroller turns on when it enters the reader's field and then runs its embedded code. The embedded code causes the gas sensor to read the resistance values of the chemiresistors connected to analog input pins to obtain a quantitative measurement of resistance. The readings from each sensor may be mapped back to an equation or a lookup table to determine which gases are present and at what concentration. The sensors are designed and selected based on their relatively orthogonal response to various vapors. Put another way, electric field power from the RFID reader is converted to current for use in reading the analog resistance of the chemoresistive sensor. The analog resistance is converted to a discrete, digital value (packetizes the very accurate analog resistance into a coded 1's and 0's digital value) by ADCs 125, 130, and 135, then that digital code is reflected back to the RFID reader via the computer 140, the transceiver 145 and the antenna 150 for recording the gas concentration value.

In various embodiments,

(1) the sensor is inexpensive (disposable)

(2) the sensor does not require a battery

(3) the sensor provides an analog signal, which is converted to a digital signal and is transmitted back to the reader as a discrete packetized value. This principle is superior to, and differs from competing concepts that rely on de-tuning an antenna, which is a concept that requires an expensive network analyzer to decode the sensor signal. Further, because these antennae are already small and have poor Q-factors, changes in the local environment can cause the same Q-reduction or frequency shift without any gas being present. Still further, the antenna resistance changes responsively to the gas, changing the quality factor Q of the antenna and shifting the frequency. The tag then stops responding to the reader because it's out of the frequency range of the reader. Antenna detuning methods can only qualitatively indicate whether gas is currently present above some threshold or not. However, the tag or reader may also just be broken. Therefore, the method is subject to poor resolution, poor accuracy, and is also extremely prone to false positives.

(4) the sensor can be made sensitive to specific gases and insensitive to other gases, including water vapor/humidity, which can be used as normalizing control.

(5) a combination (array) of several chemiresistors can be used to perform detailed gas differentiation through Principle Component Analysis (PCA).

(6) the sensor and RFID platform enables completely wireless communication between the mask of an APR, and the disposable cartridge installed on the APR.

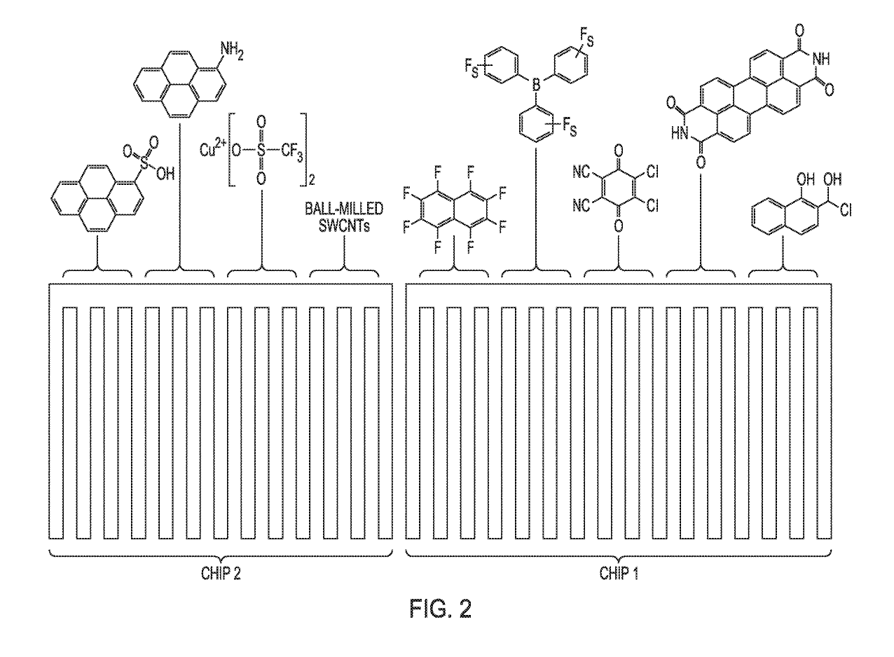

FIG. 2 is a block schematic diagram of a CCS-S sensing array 200 for multi-analyte gas sensing using PCA. Two chips 210 and 220 are shown, each having multiple chemiresistors for sensing different gasses. The chips in one embodiment may be coordinated to provide their data at different times or on different channels such that they do not interfere with each other when interrogated. The packetized data may include an identifier for each specific chemiresistor so that the corresponding resistor values may be parsed and correctly correlated by a processor that receives the signals from the chips 210 and 220.

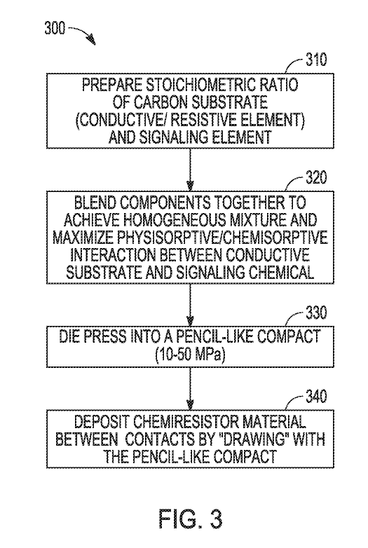

In one embodiment, chemiresistors may be fabricated utilizing the following fabrication process 300 as shown in flowchart form in FIG. 3, although other fabrication processes are also viable:

(1) A stoichiometric ratio of carbon substrate and a corresponding “signaling chemical” are stoichiometrically measured and combined to form a conductive/resistive element at 310.

(2) The mixture is extensively ball-milled to produce a carbon-based material that is evenly coated with the “signaling chemical” at the molecular level through physisorption/chemisorption. As shown at 320, the components are blended together to achieve a homogenous mixture and maximize physisorptive/chemisorptive interaction between conductive substrate and signaling chemical.

(3) the mixture is die-pressed into a pellet. In one embodiment, the mixture is die pressed into a pencil-like compact (10-50 MPa) at 330.

(4) the pellet may be used to deposit a chemiresistive trace between two ohmic contacts in a similar manner as drawing with a pencil as indicated at 340.

(5) the chemiresistive trace may be exposed to solvent vapor and the resistance between the ohmic contacts is measured.

In one embodiment, the substrate may be a carbon-based substrate used as a variable resistive trace. The trace is functionalized with the signaling chemical which may be selectively sensitive towards specific gaseous vapors. The presence of the gaseous vapor modulates the electrostatic potential of the signaling chemical and the substrate. In many cases, an oxidation-reduction response increases the sensitivity of the trace.

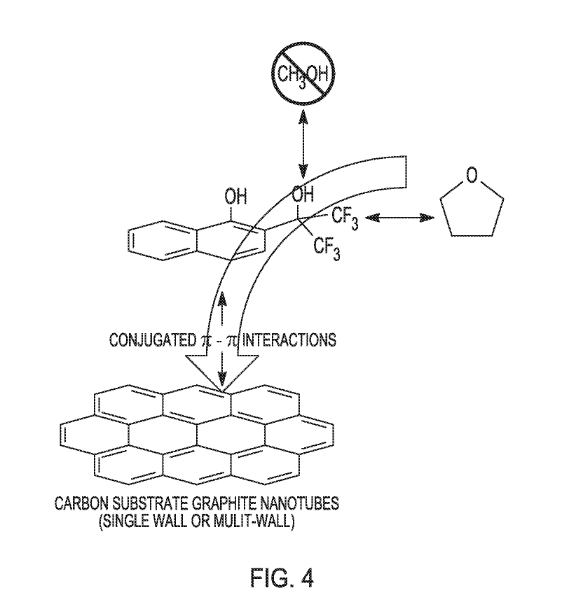

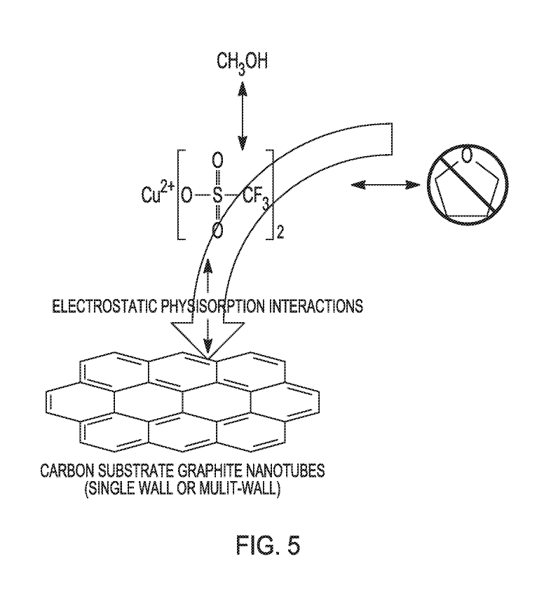

FIGS. 4 and 5 are illustrations of specific electrostatic potential modulation via vdW (van der walls) adsorptive interactions. In FIG. 4, a carbon substrate 400 is formed of carbon graphite or single wall or multiple wall nanotubes. FIG. 5 illustrates a carbon substrate 500 formed of carbon graphite or single wall or multiple wall nanotubes that interacts with different gas molecules. The substrates 400 and 500 are functionalized such that one responds to a first gas, but not a second gas, while the other responds to the second, but not the first gas.

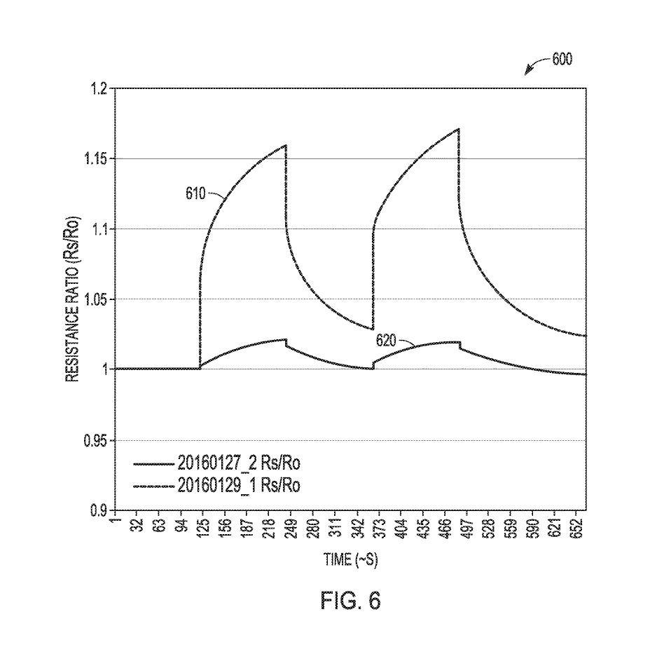

FIG. 6 is a graph 600 illustrating a resistance ratio—sensor resistor/reference resistor values for two different chemiresistors that respond to different gases. A trace 610 is representative of a water specific chemiresistor, and a trace 620 is representative of the resistance ratio corresponding to a tetrahydrofuran (THF) specific chemiresistor. The gas comprised gas at a relative humidity (RH) of 50% without THF. Trace 610 shows a much larger response than trace 620, showing that the THF specific chemiresistor was not significantly responsive to the gas.

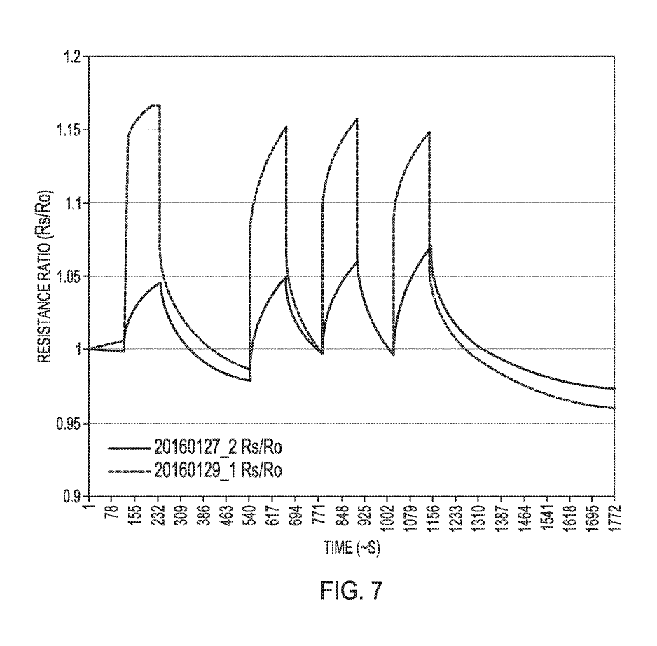

In FIG. 7, a graph 700 illustrates a resistance ratio for the same two chemiresistors, illustrated by respective traces 710 and 720, when exposed concurrently to water vapor at 50% RH with THF at 1000 ppm. Trace 710, does not change significantly from that of FIG. 6, but trace 720 showed a much larger response to the THF, further demonstrating the selectivity of the respective chemiresistors.

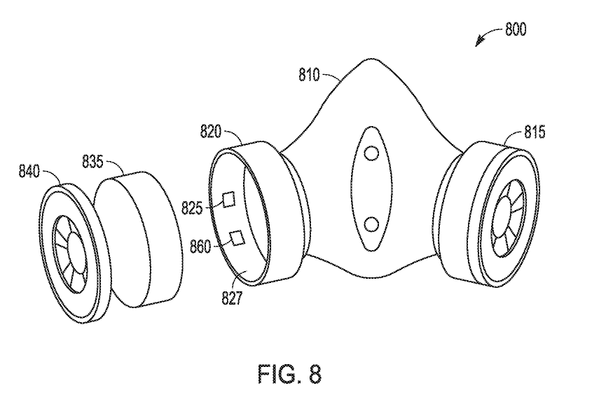

FIG. 8 is a partially exploded view of a respirator 800 having a respirator mask 810 supporting two replaceable cartridges 815, 820 with integrated end of service life indicators (ESLI) comprising battery free chemiresistive based sensor as described above which may be implemented on a chip or chipset.

One ESLI is visible in the exploded view of cartridge 820. Cartridge 815 may or may not have an ESLI, as it is likely to have a service life similar to cartridge 820. The visible ESLI is shown as a chip 825 that is supported on the inside of a cartridge body 827. The ESLI antenna is shown at 830. In one embodiment, antenna 830 may be wound around an inner perimeter of the cartridge body 827. The antenna 830 may be formed of a planar spiral of copper on Kapton substrate or aluminum on polyester substrate, such as commonly used in RFID tag antennas. An adsorbing media 835 is formed in a disk shape that fits inside the cartridge body 827. A cartridge body cap 840 secures the media 835 within the cartridge body 827.

In one embodiment, the ESLIs may be used to prevent early cartridge replacement, by providing information corresponding to the gases that the respirator has been exposed to. Prevention of early cartridge replacement can lead to fewer interruptions from premature and more frequent cartridge exchanges, reduced long term operating costs for a customer, and greater confidence of respiratory protection. Uncertainty of relying on change-out schedules can be removed, and the detection of extraordinary conductions, such as sudden gas leaks or variable gas exposure may be taken into account by establishing schedules for ESLI interrogations suitable for the environment the masks are being used in. Interrogations may be performed more often where gas exposure can be variable and extreme, such as at periods of seconds to hours in various embodiments.

In some embodiments, a low power pressure sensor 860 may be used to activate an RFID reader when breathing is detected. In one embodiment the ESLI may be buried at 90% depth in the absorbing material 835, measured from the cap 840 towards the wearer. Some preliminary results show a contactless range from the reader to the sensor of approximately 4 inches and a power transfer capability of greater than 5 mW. The chemiresistor may operate at powers of less than 50 μW.

In operation, a user may don a mask. The very low power pressure sensor 860 senses breathing of air and activates the RFID reader, which periodically queries the cartridge sensor, which indicates when vapor in the air has reached the sensor depth inside the cartridge and notifies the wearer via LED or other output notification. The sensor is thus exposed to air via the cartridge, which filters the gasses that the sensor is capable of sensing via the media 835. When the gasses are no longer adequately filtered by the media 835, the air carries the gasses to the sensor 860 where the gasses are sensed, resulting in the notification.

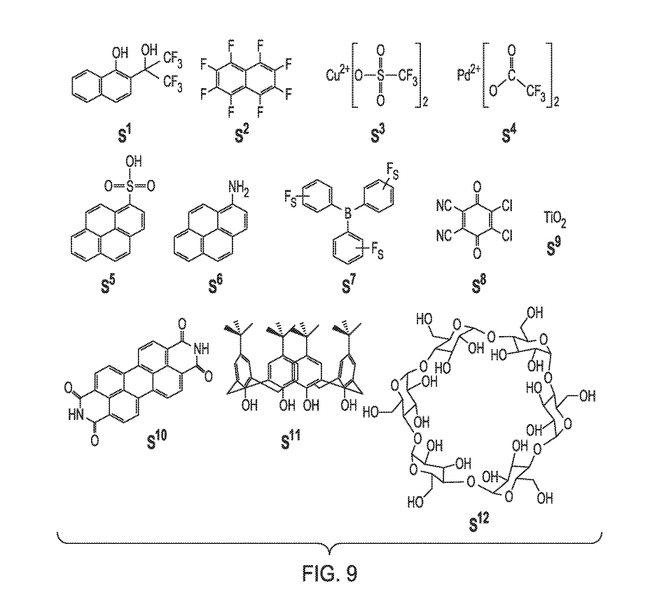

In one embodiment, the chemiresistor may be integrated into the RFID tag and read via an app on a smart phone utilizing NFC in real time. The RFID tag contains no battery or other power source, and the data may for example, be read through the wall of a glass beaker or other structure wirelessly. Several selector chemicals have been identified that demonstrate various properties of gas sensitivity and selectivity as seen in FIG. 9.

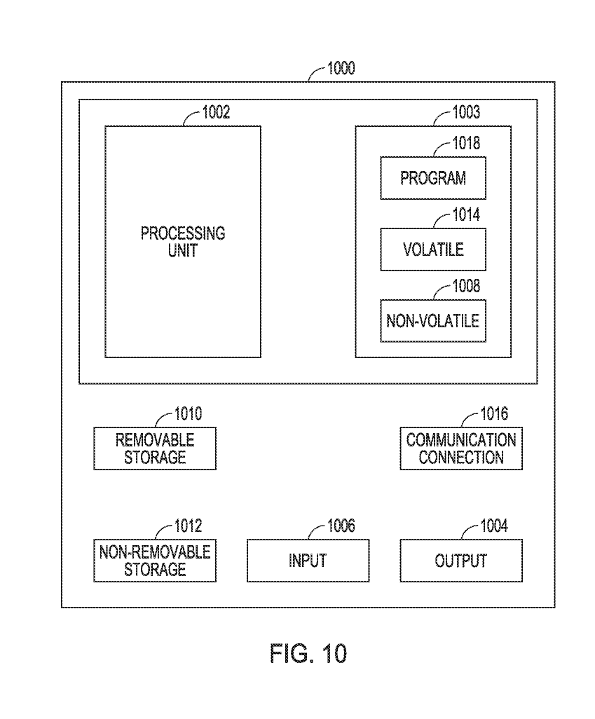

FIG. 10 is a block schematic diagram of circuitry, such as a computer 1000 to implement the interrogator, which may be powered by a button type battery or other battery, and to perform one or more methods according to example embodiments. All components need not be used in various embodiments, such as the low power computing circuitry of the sensor chip. One example computing device in the form of a computer 1000, may include a processing unit 1002, memory 1003, removable storage 1010, and non-removable storage 1012. Although the example computing device is illustrated and described as computer 1000, the computing device may be in different forms in different embodiments. For example, the computing device may instead be a simple microcontroller or other integrated circuit capable of performing the functions of the interrogator without many of the other components described below.

Memory 1003 may include volatile memory 1014 and non-volatile memory 1008. Computer 1000 may include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices capable of storing computer-readable instructions for execution to perform functions described herein.

Computer 1000 may include or have access to a computing environment that includes input 1006, output 1004, and a communication connection 1016. Output 1004 may include a display device, such as a touchscreen, that also may serve as an input device. The input 1006 may include one or more of a touchscreen, touchpad, mouse, keyboard, camera, one or more device-specific buttons, one or more sensors integrated within or coupled via wired or wireless data connections to the computer 1000, and other input devices. The computer may operate in a networked environment using a communication connection to connect to one or more remote computers, such as database servers, including cloud based servers and storage. The remote computer may include a personal computer (PC), server, router, network PC, a peer device or other common network node, or the like. The communication connection may include a Local Area Network (LAN), a Wide Area Network (WAN), cellular, WiFi, Bluetooth, or other networks.

Computer-readable instructions stored on a computer-readable storage device are executable by the processing unit 1002 of the computer 1000. A hard drive, CD-ROM, and RAM are some examples of articles including a non-transitory computer-readable medium such as a storage device. The terms computer-readable medium and storage device do not include carrier waves. For example, a computer program 1018 capable of providing a generic technique to perform access control check for data access and/or for doing an operation on one of the servers in a component object model (COM) based system may be included on a CD-ROM and loaded from the CD-ROM to a hard drive. The computer-readable instructions allow computer 1000 to provide generic access controls in a COM based computer network system having multiple users and servers.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A gas sensor comprising:
a chemiresistor having a signaling chemical responsive to a specific gas vapor to be sensed such that a resistance of the chemiresistor changes responsive to exposure to the specific gas vapor;
a data collection circuit coupled to the chemiresistor to sense the change in resistance responsive to exposure to the specific gas vapor; and
an antenna coupled to the data collection circuit to receive power from an RF interrogation signal, power the data collection circuit with the received power, and transmit a signal from the data collection circuit representative of the resistance of the chemiresistor.

2. The gas sensor of claim 1 wherein the resistance of the chemiresistor changes responsive to electrostatic interaction with the specific gas vapor.

3. The gas sensor of claim 1 wherein the change in resistance is representative of a concentration of the specific gas vapor to which the chemiresistor is exposed.

4. The gas sensor of claim 1 wherein the chemiresistor comprises carbon nanotubes and wherein the data collection circuit measures resistance of the carbon nanotubes that changes responsive to electrostatic interaction with the specific gas vapor, wherein the carbon nanotubes are single wall carbon nanotubes or multi-wall carbon nanotubes.

5. The gas sensor of claim 1 wherein the chemiresistor is configured to provide an analog output representative of electrostatic interaction with the specific gas vapor to the data collection circuit, which is configured to convert the analog output to a digital signal and output a discrete packetized value for transmission.

6. The gas sensor of claim 1 wherein the chemiresistor comprises an array of chemiresistors with multiple different signaling chemicals with functional groups capable of strong electrostatic interaction with different gas vapors to change their respective resistances.

7. The gas sensor of claim 1 wherein the chemiresistor comprises a chemoresistive trace between two ohmic contacts.

8. A method of sensing gas comprising:
exposing to air, a chemiresistor having a signaling chemical responsive to a specific gas vapor to be sensed such that a resistance of the chemiresistor changes responsive to exposure to the specific gas vapor;
sensing a specific gas vapor in the air via a data collection circuit coupled to the chemiresistor to sense the resistance that changes responsive to exposure to the specific gas vapor; and
receiving power via an RF interrogation signal at an antenna coupled to the data collection circuit and using the received power to transmit a signal from the data collection circuit representative of the resistance of the chemiresistor.

9. The method of claim 8 wherein the resistance of the chemiresistor changes responsive to electrostatic interaction with the specific gas vapor.

10. The method of claim 9 wherein the change in resistance is representative of a concentration of the specific gas vapor to which the chemiresistor is exposed.

11. The method of claim 9 wherein the chemiresistor comprises carbon nanotubes and wherein the data collection circuit measures resistance of the carbon nanotubes that changes responsive to electrostatic interaction with the specific gas vapor, wherein the carbon nanotubes are single wall carbon nanotubes or multi-wall carbon nanotubes.

12. The method of claim 8 wherein the chemiresistor is configured to provide an analog output representative of electrostatic interaction with the specific gas vapor to the data collection circuit, which is configured to convert the analog output to a digital signal and output a discrete packetized value for transmission.

13. The method of claim 8 wherein the chemiresistor comprises an array of chemiresistors with multiple different bonded signaling chemicals with function groups capable of strong electrostatic interaction with different gas vapors to change their respective resistances.

14. The method of claim 8 wherein the chemiresistor comprises a chemoresistive trace between two ohmic contacts.

15. An air purifying respirator comprising:
a filtration cartridge;
a mask coupled to the filtration cartridge, the mask configured to provide an air path from ambient to a wearer of the mask through the filtration cartridge; and
an end of service life gas sensor comprising:
a chemiresistor having a signaling chemical responsive to a specific gas vapor to be sensed such that a resistance of the chemiresistor changes responsive to exposure to the specific gas vapor;
a data collection circuit coupled to the chemiresistor to sense the change in resistance responsive to exposure to the specific gas vapor; and
an antenna coupled to the data collection circuit to receive power from an RF interrogation signal, power the data collection chipset with the received power, and transmit a signal from the data collection chipset representative of the resistance of the chemiresistor.

16. The air purifying respirator of claim 15 wherein the resistance of the chemiresistor changes responsive to electrostatic interaction with the specific gas vapor.

17. The air purifying respirator of claim 16 wherein the change in resistance is representative of a concentration of the specific gas vapor to which the chemiresistor is exposed.

18. The air purifying respirator of claim 15 wherein the chemiresistor is configured to provide an analog output representative of electrostatic interaction with the specific gas vapor to the data collection circuit, which is configured to convert the analog output to a digital signal and output a discrete packetized value for transmission.

19. The air purifying respirator of claim 15 wherein the chemiresistor comprises an array of chemiresistors with multiple different signaling chemicals with function groups capable of strong electrostatic interaction with different gas vapors to change their respective resistances.

20. The air purifying respirator of claim 15 wherein the chemiresistor comprises a chemoresistive trace between two ohmic contacts.
